(19) United States
(12) Patent Application Publication
(10) Pub. No.: US 2006/0239940 A1
(43) Pub. Date: Oct. 26, 2006

(54) Substance containing a polycation and a calcium salt

(75) Inventors: Minoru Nakayama, Kumamoto (JP); Masami Todokoro, Kumamoto (JP)

Correspondence Address: HOGAN & HARTSON LLP;IP GROUP, COLUMBIA SQUARE, 555 THIRTEENTH STREET, N.W., WASHINGTON, DC, 20004, US

(73) Assignee: CHISSO Corporation

(21) Appl. No.: 11/410,150

(22) Filed: Apr. 25, 2006

(30) Foreign Application Priority Data
Apr. 25, 2005 (JP) 2005-126553

(51) Int. Cl. A61K 8/96; A61K 8/66

(52) U.S. Cl. 424/50

(57) ABSTRACT

The invention relates to a substance containing a polycation and a calcium salt, and to a composition containing the substance. The substance and compositions containing the same exhibit adsorbability of a fungus body and adsorbability of a nucleic acid. The invention further includes an agent for adsorptive removal of a fungus body that contains the substance and a material for delivery of a nucleic acid. The invention further includes a process for obtaining the substance by removing a solvent from a mixture containing a polycation, a poorly water-soluble calcium salt, and the solvent. Preferably, the polycation is polylysine.

### BACKGROUND OF THE INVENTION

1. Field of the Invention

The invention relates to a substance containing a polycation and a calcium salt, and to a composition containing the substance.

2. Description of the Related Art

A substance obtained by binding a polycation to an insoluble carrier may be used for, 1) removing an endotoxin that causes periodontal disease from the oral cavity, 2) adsorbing a nucleic acid and introducing the adsorbed nucleic acid into a cell (DDS of a nucleic acid), or as 3) an ion exchanger.

Periodontal disease develops by the following mechanism. Pathogenic bacteria adhered on the tooth surface release LPS (lipopolysaccharides) that are endotoxins. Next, LPS binds to a specific protein that forms a periodontal tissue. Then, the conjugate further binds to a monocyte, and the monocyte is activated, resulting in occurrence of periodontal disease. From the activated monocyte, excessive amounts of cytokines such as IL-1 (interleukin 1) and TNF-α (tumor necrosis factor) or PGE₂ (prostaglandin E₂) are produced, thereby causing inflammation in the periodontal tissue. Additionally, an endotoxin is known to have an effect to prevent adhesion between the tooth and gingiva. It is generally known that, when the cytokines or PGE₂ is dispersed throughout the body by the bloodstream, inflammation may attack not only periodontal but also systemic diseases.

As described above, an endotoxin is a toxin that is deeply involved in an intraoral disease. Thus, removal of such an endotoxin is important for treatment of periodontal disease.

Several techniques for adsorptive removal of an oral endotoxin are known. There is known a technique in which a cationic polymer such as polylysine is used for an agent of adsorptive removal of an oral endotoxin (JP 2004-292357 A). The technique uses an agent of adsorptive removal formed by immobilizing a cationic polymer to a water-insoluble carrier. The agent of adsorptive removal is formed by covalently binding a cationic polymer to a water-insoluble carrier.

Additionally, a technique for removing intraoral bacteria such as Fusobacterium is also known. For example, there is exemplified a technique for adsorptive removal of intraoral bacteria using a composition for oral cavity, which is formed by immobilizing a cationic amino acid or a peptide containing it to a water-insoluble carrier (JP 06-287126 A). The composition for oral cavity is also prepared by covalently immobilizing a peptide or the like to a polysaccharide-based water-insoluble carrier, so that the production method is cumbersome.

Drug delivery system (DDS) is an administration method that enables persistence of a drug efficacy, reduction in dosage, and reduction of side effects or the like. As DDS for a hormone therapeutic drug, Leuplin® (manufactured by Takeda Pharmaceutical Company Limited.) is famous, which is produced by incorporating leuprorelin acetate in a microcapsule including a lactate polymer that is a biodegradable polymeric compound as a carrier.

As DDS for a nucleic acid, virus vectors and non-viral vectors are known. A polycation (in particular, polyethyleneimine, α-polylysine, lysine dendrimer, chitosan, or the like) is applied to the nonviral vector. For example, it is known that a conjugate of a polycation and a protein is applied as a virus vector (JP 06-505980 A). However, there are problems such as strong cytotoxicity, low efficiency of gene introduction, and low expression efficiency, although those non-viral vectors have no problem unique to viruses.

The ion exchange method is used for removing contaminants such as heavy-metal compounds, ammoniacal nitrogen, and phosphate compounds or for collecting and reusing them as valuable resources. In view of the circumstances where the wastewater standards will be strengthened, the ion exchange method will be adopted for wastewater treatments and the like.

The ion exchange method is performed using an ion exchanger, which is a substance that undergoes an ion exchange reaction. The ion exchanger is obtained by binding a substance having ion exchangeability (for example, polycation) to an insoluble carrier. Examples of the ion exchanger include polysaccharide carriers as typified by cellulose ion exchangers and synthetic resin carriers.

A polycation that is a substances having ion exchangeability is required to bind to a polysaccharide that is an insoluble carrier at a hydroxyl group existing in the polysaccharide via a crosslinking group. Here, the crosslinking is performed using epichlorohydrin or the like as a crosslinker.

Further, an ion exchanger including a synthetic resin as an insoluble carrier is produced by polymerizing a monomer composition containing a cationic monomer (a monomer that forms a polycation).

As described above, substances obtained by binding a polycation to an insoluble carrier have various applications.

In general, all the substances are often produced by immobilizing a ligand having adsorbability or ion exchangeability (i.e., polycation) to a fine particle that is an insoluble carrier.

The fine particle that is an insoluble carrier is often synthesized from a polysaccharide gel such as silica gel or cellulose, or a synthetic polymer substance such as acrylic-based, vinyl-based, styrene-based, nylon or epoxy-based substance.

A polycation that is a ligand having adsorbability or ion exchangeability is a water-soluble polymer that shows basic. Usually, to use a polycation as an adsorbent or ion exchanger, it is required to be immobilized to a fine particle that is an insoluble carrier (see, for example, Liq. Chrom & Rel. Technol., 25 (4), 2002, 601-614). In general, a polycation is immobilized to an insoluble carrier by reacting with an insoluble carrier in which an active group having reactivity to an amino group is introduced and by binding covalently to the insoluble carrier. Examples of a known active group to be introduced in an insoluble carrier and to be allowed to react with an amino group include epoxy, aldehyde, chloromethyl, carboxyl, and acid chloride groups, and the like.

On the other hand, it is known that concomitant use of a polycation and a plant extract improves the glucosyltransferase (GTF) inhibitory activity of the plant extract, resulting in improvement of an effect to suppress formation of dental plaque (JP 2004-196756 A.)

### SUMMARY OF THE INVENTION

The invention provides a new substance containing a polycation and a carrier that can immobilize the polycation.

In particular, the invention provides a carrier that is not required to be activated to immobilize a polycation, has a good image for consumers because it contains natural substances as primary ingredients, and exerts adsorbability of a bacterial toxin by combining with a polycation, and may be used as a DDS material or an ion exchanger.

Further, the invention provides a substance that contains a polycation and a carrier and has more improved adsorbability of a bacterial toxin or adsorbability of a nucleic acid than before.

It has been observed that a product obtained by mixing a polycation and calcium salts can immobilize a polycation and bind to the immobilized polycation even in an aqueous solution having a low salt concentration without releasing it. Accordingly, a calcium salt is used as a carrier for a polycation. That is, the invention includes:

(1) A product obtained by removing a solvent from a mixture containing a polycation, a poorly water-soluble calcium salt, and the solvent.

(2) A product according to item (1), wherein the mixture is obtained by mixing a polycation, a water-soluble calcium salt, a compound to convert the water-soluble calcium salt into a poorly water-soluble calcium salt, and the solvent.

(3) A product according to item (1), wherein the polycation is a free polycation.

(3) A product according to item (1), wherein the polycation is a polyamino acid.

(4) A product according to item (1), wherein the polycation is polylysine.

(5) A product according to item (1), wherein the polycation is ε-polylysine.

(6) A composition for removing an oral endotoxin, which comprises a product obtained by removing a solvent from a mixture containing a polycation, a poorly water-soluble calcium salt, and the solvent.

(7) A composition for introducing an adsorbed nucleic acid into a cell, the composition comprising a product obtained by removing a solvent from a mixture containing a polycation, a poorly water-soluble calcium salt, and the solvent, and adsorbing a nucleic acid.

(8) A method of producing a composition for removing an oral endotoxin, the composition containing a product obtained by removing a solvent from a mixture containing a polycation, a poorly water-soluble calcium salt, and the solvent, the method comprising a step of incorporating the product obtained by removing the solvent from the mixture containing the polycation, the poorly water-soluble calcium salt, and the solvent.

(9) A method of producing a composition for introducing an adsorbed nucleic acid into a cell, the composition containing a product obtained by removing a solvent from a mixture containing a polycation, a poorly water-soluble calcium salt, and the solvent, and adsorbing a nucleic acid,the method comprising a step of incorporating the product obtained by removing the solvent from the mixture containing the polycation, the poorly water-soluble calcium salt, and the solvent.

(10) A product according to item [1], wherein the poorly water-soluble calcium salt is a mixture of one kind or two or more kinds selected from the group consisting of CaF₂, CaSO₄, and a salt represented by the following composition Formula 1 or 2:
H_{w}CaₓO_{y}P_{z}.A_{b} Formula 1:
wherein H, Ca, O, and P represent hydrogen, calcium, oxygen, and phosphorus respectively; w represents an integer of 0 or more; x, y, and z represent integers of 1 or more respectively; A is an element or a composition other than hydrogen, calcium, oxygen, and phosphorus, which does not prevent poor solubility of calcium, for example, fluorine; and b represents an integer of 0 or more.
H_{w}CaₓO_{y}C_{z}.A_{b} Formula 2:
wherein H, Ca, O, and C represent hydrogen, calcium, oxygen, and carbon respectively; w represents an integer of 0 or more; and x, y, and z represent integers of 1 or more respectively; A is an element or a composition other than hydrogen, calcium, oxygen, and carbon, and which does not prevent poor solubility of calcium, for example, magnesium; and b represents an integer of 0 or more.

The product of the invention has adsorbability of a bacterial toxin or adsorbability of a nucleic acid, so that a composition containing the product of the invention can be used as an agent of adsorptive removal of a bacterial toxin or as a carrier for delivery of a nucleic acid.

### DETAILED DESCRIPTION OF THE INVENTION

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The product of the invention is a substance obtained by removing a solvent from a mixture containing a polycation, a poorly water-soluble calcium salt, and the solvent (hereinafter, the mixture is referred to as “mixed solution”) and may be, for example, a dry powder.

The product of the invention may be a mixed salt of a polycation and a poorly water-soluble calcium salt or may be a mixture of a polycation and a poorly water-soluble calcium salt. As described below, the product of the invention does not elute (or hardly elutes) a polycation when even suspended in an aqueous solution having a low salt concentration. Therefore, at least part of the polycation is considered to be present as a mixed salt with a poorly water-soluble calcium salt.

Additionally, as described below, the endotoxin adsorption efficiency of the product of the invention is different from that of a mere mixture of a polycation and a poorly water-soluble calcium salt and that of a mere mixture of a polycation hydrochloride and a poorly water-soluble calcium salt, so that the product of the invention is considered to be not a mere mixture but a substance containing polylysine immobilized by a poorly water-soluble calcium salt.

A polycation to be contained in a mixed solution may be a polymeric compound having a cationic group or a group that may become a cationic group, and an aqueous solution of a free polycation shows basic. Examples of the group that may become a cationic group include an amino group, an imino group, and the like.

Examples of polycation include: polyamino acid such as polylysine, polyornithine, polyhistidine, polyarginine, polytryptophan, poly-2,4-diaminobutyric acid, poly-2,3-diaminopropionic acid, protamine, and polypeptide having at least one or more kinds of amino acid residues in a polypeptide chain selected from the group consisting of lysine, histidine, arginine, tryptophan, ornithine, 2,4-diaminobutyric acid and 2,3-diaminopropionic acid; polyamine such as polyallylamine, polyvinylamine, a copolymer of allylamine and diallylamine, and polydiallylamine; and polyimine such as polyethyleneimine.

More preferable examples of the polycation include a polyamino acid, and further preferable examples thereof include polylysine and the like. Polylysine may be ε-polylysine, α-polylysine, or a mixture of α- and ε-polylysines, but ε-polylysine is preferable. A preferable polymerization degree of ε-polylysine is 25 to 35. Additionally, the average degree of polymerization of ε-polylysine is preferably about 31. ε-Polylysine is most preferably used, for the reason that it is more inexpensive than a chemically synthesized product because of fermentative production and it is high safety because it has been used as a food additive for a long time, and the like. Further, polylysine may be a dendrimer of lysine.

A polycation to be contained in a mixed solution is preferably a free polycation. That is, a polycation may become a salt by reacting with an acidic substance, but it is preferable that a polycation to be used in the invention is not such a salt. The reasons are as follows:

(1) Use of a free polycation can increase the polycation content in a product compared with the case of using a polycation salt. The fact is shown also in Examples described below.

(2) A product obtained by using a free polycation hardly releases (for example, elutes) a polycation compared with a product obtained by using a polycation hydrochloride.

(3) A product obtained by using a free polycation may have higher adsorbability of an endotoxin than a product obtained by using a polycation hydrochloride even if the polycation contents are the same. The fact is shown also in Examples described below.

Those reasons are unclear, but it may be considered that a free polycation is easy to form a mixed salt with a calcium salt, so that the calcium salt immobilizes more polycation and does not release the immobilized polycation, resulting in improvement of the adsorbability.

A poorly water-soluble calcium salt contained in a mixed solution is an inorganic calcium salt composed of a calcium ion and an ion that forms a poorly water-soluble calcium salt by binding to the calcium ion. The poorly water-soluble calcium salt contained in a mixed solution may be only one kind of salt or a combination of two or more salts.

Examples of the poorly water-soluble calcium salt include a mixture of one or two or more selected from the group consisting of CaF₂, CaSO₄, and a salt represented by the following compositions of Formula 1 or 2.
H_{w}CaₓO_{y}P_{z}.A_{b} Formula 1:

In the compositions of Formula 1, H, Ca, O, and P represent hydrogen, calcium, oxygen, and phosphorus respectively. “w” represents an integer of 0 or more, while x, y, and z represent integers of 1 or more, respectively. Additionally, A is an element or a composition other than hydrogen, calcium, oxygen, and phosphorus, and examples thereof include fluorine, which does not prevent poor solubility of calcium. “b” represents an integer of 0 or more.

Examples of the poorly water-soluble calcium salt represented by compositions of Formula 1 include calcium phosphates (including hydroxyapatite), and specific examples thereof include Ca₃(PO₄)₂, CaHPO₄, Ca₁₀(PO₄)₆(OH)₂, calcium diphosphate (calcium pyrophosphate) Ca₂P₂O₇, calcium dihydrogen diphosphate (calcium dihydrogen pyrophosphate) CaH₂P₂O₇, pentacalcium trisphosphate hydroxide (Ca₅(OH)(PO₄)₃), fluoroapatite Ca₁₀(PO₄)₆F₂, a mixture of one or two or more selected from the group consisting of such substances, and the like. Preferable examples include Ca₃(PO₄)₂, Ca₁₀(PO₄)₆(OH)₂, and a mixture of Ca₃(PO₄)₂ and Ca₁₀(PO₄)₆(OH)₂.
H_{w}CaₓO_{y}C_{z}.A_{b} Formula 2:

In the compositions of Formula 2, H, Ca, O, and C represent hydrogen, calcium, oxygen, and carbon, respectively. “w” represents an integer of 0 or more, while x, y, and z represent integers of 1 or more, respectively. Additionally, A is an element or a composition other than hydrogen, calcium, oxygen, and carbon, and examples thereof include magnesium, which does not prevent poor solubility of calcium. “b” represents an integer of 0 or more.

Examples of the poorly water-soluble calcium salt represented by compositions of Formula 2 include calcium carbonates, and specific examples thereof include CaCO₃, and CaMg(CO₃)₂. Preferable examples include CaCO₃.

A mixed solution containing a polycation, a poorly water-soluble calcium salt, and a solvent may be (a) a mixed solution prepared by mixing a polycation and a poorly water-soluble calcium salt in a solvent (hereinafter, referred to as “mixed solution A”) or (b) a mixed solution prepared by mixing a polycation, a water-soluble calcium salt, and a compound to convert the water-soluble calcium salt into a poorly water-soluble calcium salt, in a solvent (hereinafter, referred to as “mixed solution B”).

The polycation and poorly water-soluble calcium salt to be mixed in the mixed solution A are the same as the above-described polycation and poorly water-soluble calcium salt.

The solvent in the mixed solution A is preferably an aqueous solvent that is hard to dissolve a poorly water-soluble calcium salt and is easy to dissolve a polycation.

Examples of the aqueous solvent include water, ethanol, isopropyl alcohol, a mixed solution of water/ethanol, methanol, and the like. Preferable aqueous solvent is water, ethanol, isopropyl alcohol, and a mixed solution of water/ethanol, and more preferable aqueous solvent is water.

In a preparation of the mixed solution A, the poorly water-soluble calcium salts content in the solvent is not particularly limited as long as the polycation solution and poorly water-soluble calcium salts can be mixed uniformly. The poorly water-soluble calcium salts content is preferably equal to three-fold amount. Specifically, the poorly water-soluble calcium salts content is preferably 0.1 to 1 g/mL, further preferably 0.2 to 1 g/mL. On the other hand, the polycation content in the solvent is preferably 1 to 330 mg/mL, further preferably 1 to 50 mg/mL.

Additionally, with regard to the ratio between the poorly water-soluble calcium salts and the polycation to be mixed in the mixed solution A, the amount of the polycation is not particularly limited and may be excess, but the weight ratio is preferably 0.3:1 to 200:1, further preferably 20:1 to 200:1 in view of washing with water for removing an excess of the polycation.

Further, the particle size of a poorly water-soluble calcium salt to be mixed in the mixed solution A is preferably 1 to 100 μm. The particle size of a poorly water-soluble calcium salt may be approximately equal to that of powder of calcium phosphate (commercially available food additive). In the case of using the salt only in the oral cavity, the particle size is preferably approximately several tens of μm. Adjustment of the particle size enables adjustment of the polycation content in the resultant product.

Mixing in the preparation of the mixed solution A is preferably performed so that the poorly water-soluble calcium salt or the resultant product can be uniformLy dispersed. The means for mixing is not particularly limited, but mixing may be performed using, for example, a stirring machine, homomixer, dispersing machine, or the like.

Mixing in the preparation of the mixed solution A may be performed at a temperature where a polycation does not degrade, and it is preferably performed at a temperature of between approximately 10 to approximately 60° C. The time for mixing is not particularly limited, but is preferably between approximately 1 to approximately 8 hours.

The polycation to be mixed in the mixed solution B is the same as the above-described polycation.

The water-soluble calcium salt to be mixed in the mixed solution B is not particularly limited as long as it is dissolved in a solvent, but it is preferably an inorganic calcium salt. Additionally, the water-soluble calcium salt forms the poorly water-soluble calcium salt in the mixed solution B, and the formed poorly water-soluble calcium salt is deposited in the solvent.

Examples of the water-soluble calcium salt include calcium chloride, Ca(H₂PO₄)₂, calcium acetate, calcium lactate, calcium bromide, calcium iodide, and calcium nitrate, and the like. Calcium chloride, Ca(H₂PO₄)₂, calcium acetate, and calcium lactate are preferable water-soluble calcium salts.

The “compound to convert a water-soluble calcium salt into a poorly water-soluble calcium salt” to be mixed in the mixed solution B is not particularly limited as long as it is a compound that generates an ion that forms a poorly water-soluble calcium salt by binding to a calcium ion. Examples of the ion include phosphate ion, sulfate ion, and the like. Examples of the compound that generates phosphate ion include sodium phosphate, ammonium phosphate, phosphoric acid, sodium hydrogen phosphate, sodium dihydrogen phosphate, potassium phosphate, and the like.

The solvent in the mixed solution B is preferably a solvent that dissolves a water-soluble calcium salt and a polycation, and it is preferably an aqueous solvent that can separate out (precipitate) a substance generated by mixing.

Examples of the aqueous solvent include water, ethanol, isopropyl alcohol, a mixed solution of water/ethanol, methanol, and the like. Preferable aqueous solvent is water, ethanol, isopropyl alcohol, and a mixed solution of water/ethanol, and more preferable aqueous solvent is water.

In a preparation of the mixed solution B, the water-soluble calcium salt content in the mixed solution is preferably approximately 0.05 to approximately 0.5 mol/l. On the other hand, the content of the compound to convert a water-soluble calcium salt into a poorly water-soluble calcium salt is preferably approximately 0.1 to approximately 1.0 mol/L.

In the preparation of the mixed solution B, the ratio between the water-soluble calcium salt and the compound to convert a water-soluble calcium salt into a poorly water-soluble calcium salt to be mixed is preferably approximately 2:1 to approximately 1:2 (in terms of molar ratio).

The polycation content contained in the mixed solution B is preferably approximately 0.01 to approximately 0.10 g/l. Additionally, in the preparation of the mixed solution B, the polycation to be mixed is preferably approximately 20 to approximately 2,000 g/mol with respect to the calcium salt.

The mixed solution B may be prepared by mixing the above-described three kinds of ingredients in a solvent, and the procedure (such as the order to add each ingredient to the solvent) is not particularly limited.

Examples of a preferable procedure include a procedure to:

(a) add a polycation to a solution containing a water-soluble calcium salt and a compound to convert the water-soluble calcium salt into a poorly water-soluble calcium salt;

(b) add a water-soluble calcium salt and a compound to convert the water-soluble calcium salt into a poorly water-soluble calcium salt to a homogeneous solution containing a polycation;

(c) add a compound to convert a water-soluble calcium salt into a poorly water-soluble calcium salt to a solution that may be either a homogeneous solution containing a polycation and a water-soluble calcium salt or a solid-liquid mixture; or

(d) add a water-soluble calcium salt to a solution containing a polycation and a compound to convert the water-soluble calcium salt into a poorly water-soluble calcium salt.

Moreover, in the case that a system formed by mixing the three kinds of ingredients is acidic, if necessary, the system is preferably neutralized to neutral to basic (pH 7 to 9). As a neutralizing agent, potassium hydroxide or the like may be used. Given that mixing and heating of only potassium hydroxide and polylysine may induce hydrolysis, it is preferable that potassium hydroxide be not added to a solution containing only a polycation.

In a manner similar to mixing in the preparation of the mixed solution A, mixing in the preparation of the mixed solution B is preferably performed so that a water-soluble calcium salt, a compound to convert the water-soluble calcium salt into a poorly water-soluble calcium salt, and a precipitate can be uniformly dispersed. The means for mixing is not particularly limited and is the same as that in the preparation of mixed solution A.

In the preparation of the mixed solution B, mixing is preferably performed at a temperature of approximately 20 to approximately 95° C. Given that a polycation may degrade at a high temperature of 60° C. or more, it is preferable that mixing of a polycation is performed at a temperature of approximately 10 to approximately 60° C. The time for mixing is preferably approximately 1 to approximately 10 hours.

The product of the invention is obtained by removing a solvent contained in the above-described mixed solution, and here, the solvent is removed by filtration and/or distilling away the solvent. Here, at least only part of the solvent has to be removed, but substantially all the solvent is preferably removed. Conceivably, the poorly water-soluble calcium salt could more firmly immobilize the polycation by removing the solvent.

The product obtained by removing a solvent from a mixed solution can be subjected to a treatment such as washing or drying. For example, when the product is washed with water, impurities (for example, a polycation that does not bind to a poorly water-soluble calcium salt) may be removed. Additionally, when the product is dried, it can be made dry powder. Drying may be performed by any means such as heated-air drying, reduced-pressure drying (including vacuum drying), freeze-drying, or spray drying.

The product of the invention contains a poorly water-soluble calcium salt and a polycation, and the polycation content is preferably approximately 0.01% or more by weight, more preferably approximately 0.05% or more by weight, further preferably approximately 0.2% or more by weight with respect to the entire product.

In producing the product, the polycation content is adjusted by appropriately selecting the kind and amount of the polycation, kind and particle size of the calcium salt, mixing conditions, and the like. For example, in the case of using a free polycation, the content is higher than that in the case of using a polycation salt (for example, hydrochloride). The polycation content in the product of the invention is calculated from the nitrogen content determined by the Kjeldahl method.

The product of the invention contains a poorly water-soluble calcium salt and a polycation, which is characterized in that it does not elute or hardly elutes calcium even if it is suspended in an aqueous solution having a low salt concentration (for example, aqueous solution having approximately the same salt concentration as that of a biological system, i.e., normal saline solution) (approximately 0.15 mol/L). In particular, the product of the invention suspended in a buffer having a salt concentration corresponding to that of saliva is hard to elute a polycation.

Therefore, at least part of the polycation is considered to form a mixed salt with the water-soluble calcium salt.

The product of the invention has a property to adsorb a bacterial toxin (adsorbability of a bacterial toxin). Preferable examples of the bacterial toxin include an endotoxin that is an intraoral toxin. Therefore, the product of the invention is preferably contained in a composition for adsorptive removal of a bacterial toxin (agent of adsorptive removal). The composition may be applied to, for example, a cleaning agent for oral cavity, chewing gum, and the like.

The product of the invention has a property to adsorb a nucleic acid (adsorbability of a nucleic acid). Examples of the nucleic acid include deoxyribo nucleic acid and ribonucleic acid. Therefore, the product of the invention is preferably contained as an ingredient of a composition for adsorption of a nucleic acid and delivery of the adsorbed nucleic acid into a cell (nucleic acid delivery). The composition may be applied to, for example, cosmetic products, drug delivery carriers, carriers for gene introduction, carriers for diagnostic agents, and the like.

Composition of the Invention

The composition of the invention is characterized by containing the above-described product of the invention, and further contains any ingredient within a scope which does not severely ruin the effects of the invention. Any ingredient is appropriately selected according to use or dosage form of the composition.

The product of the invention has adsorbability of a bacterial toxin (in particular, intraoral toxin) or adsorbability of a nucleic acid, so that the composition of the invention is used for adsorptive removal of fungus body or for delivery of a nucleic acid. In the case that the composition of the invention is used for adsorptive removal of an oral toxin, it is used as a cleaning agent for oral cavity (irrigator solution, mouthwash, dentifrice, or the like) or chewing gum. In the case that the composition of the invention is used for deliver of a nucleic acid, it is used as a cosmetic product, drug delivery carrier, carrier for gene introduction, carrier for a diagnostic agent, or the like.

Production Method of the Invention

The production method of the invention is a method of producing the above-described composition, which includes: (1) a step for obtaining a product obtained by removing a solvent from a mixture containing a polycation, a poorly water-soluble calcium salt, and the solvent and (2) a step for incorporating the resultant product together with an optional ingredient. As described above, the optional ingredient may be appropriately selected according to use of the composition.

### EXAMPLES

The following example are for illustrative purposes only and are not intended, nor should they be interpreted to, limit the scope of the invention.

### Example 1

A polycation was added to a mixture of calcium salts and a solvent heated to 40° C., and the mixture was stirred with a magnetic stirrer (40° C., 1 hour). The solvent in the resultant reaction solution was distilled away with an evaporator, followed by vacuum drying (40° C., 18 hours), to thereby yield dry powder. The dry powder was washed twice with water having a volume 5 times that of the dry powder, followed by vacuum drying (40° C., 18 hours) again, to thereby yield dry powder. The polycation content in the resultant powder was calculated from the total nitrogen content determined by the Kjeldahl method.

Table 1 shows the kinds and amounts of used poorly water-soluble calcium salts, the kinds and amounts of used solvents, and the kinds and amounts of used polycations.

The descriptions in the columns for names of polycations are abbreviations of the following compounds: EPL: ε-polylysine (average degree of polymerization: 31) (product manufactured by CHISSO CORPORATION); APL: α-polylysine (average degree of polymerization: 3,700) (a product manufactured by Toray Industries, Inc.); PAA: polyallylamine (average degree of polymerization: 180) (product manufactured by Nitto Boseki Co., Ltd.); PEI: polyethyleneimine (average degree of polymerization: 900) (product manufactured by Wako Pure Chemical Industries, Ltd.

TABLE 1

| | Poorly Water-Soluble | | | | | | | |
|---|---|---|---|---|---|---|---|---|
| | Calcium Salt | | Solvent | | Polycation | | Dry | Polycation |
| | Name or | Amt. | | Volume | | Amount | Powder | Content in dry |
| No. | composition | (g) | Name | (mL) | Name | (g) | Yield (g) | powder (w/w %) |
| CP1 | Ca₃(PO₄)₂ | 40.0 | Water | 60 | EPL | 0.250 | 38.0 | 0.50 |
| CP2 | Ca₃(PO₄)2 | 29.0 | Water | 60 | EPL | 0.250 | 49.0 | 0.60 |
| | CaHPO₄ | 11.0 | | | | | | |
| CP3 | Ca₃(PO₄)₂ | 10.0 | Water | 40 | PAA | 0.062 | 9.6 | 0.57 |
| CP4 | Ca₃(PO₄)₂ | 10.0 | Water | 40 | PEI | 0.062 | 9.3 | 0.55 |
| CP5 | Ca₃(PO₄)₂ | 10.0 | Water | 40 | APL | 0.062 | 9.4 | 0.55 |
| CP6 | Ca₃(PO₄)₂ | 20.0 | Water | 30 | EPL | 0.323 | 19.4 | 0.36 |
| | | | | | hydrochloride | | | |
| CP7 | Ca₃(PO₄)₂ | 14.5 | Water | 30 | EPL | 0.323 | 19.2 | 0.73 |
| | CaHPO₄ | 5.5 | | | hydrochloride | | | |
| HA1 | Hydroxyapatite | 3.4 | Water | 34 | EPL | 11.000 | 3.3 | 0.10 |
| HA2 | Hydroxyapatite | 10.0 | Water | 40 | PAA | 0.125 | 7.8 | 0.61 |
| HA3 | Hydroxyapatite | 10.0 | Water | 40 | PEI | 0.125 | 8.0 | 0.58 |
| HA4 | Hydroxyapatite | 10.0 | Water | 40 | APL | 0.125 | 7.6 | 0.55 |
| CC1 | CaCO₃ | 10.0 | Water | 100 | EPL | 10.000 | 10.0 | 0.05 |
| CC2 | CaCO₃ | 10.0 | Ethanol | 100 | EPL | 10.000 | 10.0 | 0.10 |

Table 1 reveals that each of the dry powder contains a polycation. Moreover, through a comparison between the case of using a free polycation (CP1) and the case of using a polycation salt (CP6), the polycation content in the dry powder of CP1 is found to be higher than that of CP6, although CP6 uses 2-fold molar excess of the polycation with respect to a poorly soluble calcium salt. That is, it is found that using a free polycation enables increasing the polycation content in a product compared with the case of using a polycation salt.

### Example 2

1 L of 0.01 mol/L aqueous sodium phosphate solution was heated to 95° C. 0.05 mol/L aqueous calcium chloride solution (1L) and 0.5 mol/L aqueous disodium hydrogen phosphate solution (1.5 L) were delivered dropwise over approximately 2 hours and 30 minutes.

Thereafter, 1 mol/L aqueous potassium hydroxide solution was delivered dropwise (in the amount described in Table 2) over approximately 40 minutes, and then the mixture was further stirred for 30 minutes. The mixture was allowed to cool to 40° C., and 25% by weight of ε-polylysine was added (in the amount described in Table 2), followed by stirring for 3 hours. The resultant reaction solution was filtered, and was washed with water until the filtrate became neutral. The substances obtained by filtration were subjected to vacuum drying (60° C., 18 hours), to thereby yield dry powder. The polycation content in the dry powder was calculated from the total nitrogen content determined by the Kjeldahl method.

TABLE 2

| | 25 w/w % | Potassium | Dry | Polycation |
|---|---|---|---|---|
| | ε-Polylysine | Hydroxide | Powder | Content in Dry |
| No. | (L) | (1 mol/L) (L) | Yield (g) | Powder (w/w %) |
| HA5 | 0.5 | 0.30 | 102 | 0.1 |
| HA6 | 1.0 | 0.15 | 80 | 0.6 |

### Example 3

### Endotoxin Adsorbability

Buffer A having the following composition was allowed to incorporate each of 380,000 EU/mL, 1,470,000 EU/mL, 1,530,000 EU/mL, or 1,260,000 EU/mL of an endotoxin (Lipopolysaccharide from Escherichia Coli 0111: B4, Sigma), to thereby yield Solution 1 (380,000 EU/mL), Solution 2 (1,470,000 EU/mL), Solution 3 (1,530,000 EU/mL), and Solution 4 (1,260,000 EU/mL).

Buffer A: NaCl, 15 mmol; KCl, 20 mmol; NaHCO₃, 15 mmol; NaH₂PO₄, 13.2 mmol (pH 6.7).

To the resultant solutions 1, 2, 3, and 4 (10 mL each) were added the dry powder (0.2 g) obtained in Examples 1 and 2, followed by shaking at 37° C. for 2 hours. Thereafter, the mixtures were filtered with a filter having a pore size of 0.45 μm, and the concentrations of the endotoxin in the filtrate were determined.

The endotoxin was quantified using a kit for determining endotoxins (Endospecy) manufactured by Seikagaku Corporation. The results are shown in Table 3.

### Comparative Example 1

### Endotoxin Adsorbability

The concentrations of the endotoxin were determined by the same experiment as Example 3 except that the dry powder obtained in Examples 1 and 2 were replaced by a calcium salt containing no polycation (Ca₃(PO₄)₂, CaHPO₄, or hydroxyapatite), a mere mixture of a calcium salt and a polycation (Sample number Ref1: Ca₃(PO₄)₂ (0.2 g)+EPL (0.001 g) mixture (polycation content 0.5 w/w %)), and a mere mixture of a calcium salt and a polycation hydrochloride (Sample number Ref2: Ca₃(PO₄)₂ (0.2 g)+EPL hydrochloride (0.001 g) mixture (polycation content 0.5 w/w %)) in Example 3. The results are shown in Table 3.

TABLE 3

| | Sample | Initial ET | Residual ET | ET removal |
|---|---|---|---|---|
| | Weight | Concentration | Concentration | Efficiency |
| Sample | (g) | (EU/mL) | (EU/mL) | (%) |
| CP1 | 0.2 | 1,470,000 | 104,000 | 92.9 |
| CP2 | 0.2 | 1,470,000 | 1,100 | 99.9 |
| CP6 | 0.2 | 1,530,000 | 353,000 | 76.9 |
| CP7 | 0.2 | 1,530,000 | 262,000 | 82.9 |
| Ca₃(PO₄)₂ | 0.2 | 1,470,000 | 1,117,200 | 24.0 |
| CaHPO₄ | 0.2 | 1,470,000 | 984,900 | 33.0 |
| HA5 | 0.2 | 380,000 | 10,000 | 97.4 |
| HA6 | 0.2 | 380,000 | 2,500 | 99.3 |
| Hydroxyapatite | 0.2 | 380,000 | 92,000 | 75.8 |
| Ref1 | 0.2 | 1,530,000 | 574,000 | 62.5 |
| Ref2 | 0.2 | 1,260,000 | 525,000 | 58.3 |

In Table 3, ET is an abbreviation of endotoxin.

As shown in Table 3, the endotoxin removal efficiencies in the cases of using the dry powder obtained in Examples 1 and 2 (CP1, 2, 6, 7, HA2, 3: powders containing a polycation and a calcium salt) are significantly higher than those in the cases of using the calcium salt containing no polycation. That is, the dry powder obtained in Examples 1 and 2 were found to have high adsorbabilities of the endotoxin.

Additionally, the endotoxin removal efficiency in the case of adding the product of the invention (CP1) was found to be higher than that in the case of adding a calcium salt and a polycation (Ref1). The endotoxin removal efficiency in the case of adding the product of the present invention (CP6) was found to be higher than that in the case of adding a calcium salt and a polycation hydrochloride (Ref2). That is, the dry powder obtained in the invention were found not to be mere mixtures and to have higher adsorbabilities of the endotoxin than those of mere mixtures.

When the mere mixture of a calcium salt and a polycation (Ref1) and the mere mixture of a calcium salt and a polycation hydrochloride (Ref2) have the same polycation content, they have approximately the same endotoxin removal efficiencies. While even when the mixture of a calcium salt and a polycation of the invention (CP1) and the mixture of a calcium salt and a polycation hydrochloride of the present invention (CP6) have approximately the same polycation content, they have different endotoxin removal efficiencies. That is, the product obtained by using a free polycation was found to have higher adsorbability of the endotoxin than that obtained by using a polycation hydrochloride.

### Example 4

### Nucleic Acid Adsorbability

To 10 mL of 0.02 mol/l sodium phosphate buffer (pH 6.7) containing 0.04 mg/mL nucleic acid (deoxyribo nucleic acid, derived from salmon semen, Wako Pure Chemical Industries, Ltd.) was added the powders obtained in Example 1, followed by shaking at 25° C. for 2 hours. Thereafter, the mixtures were filtered with a filter having a pore size of 0.45 μm, and the concentrations of a nucleic acid in the filtrate were determined.

Specifically, the ultraviolet absorptions at a wavelength of 260 nm were determined, and the concentrations of the nucleic acid were calculated from the determination results and created standard curves. The results are shown in Table 4.

### Comparative Example 2

### Nucleic Acid Adsorbability

The concentrations of the nucleic acid were determined by the same experiment as Example 4 except that, in Example 4, calcium salts containing no polycation (Ca₃(PO₄)₂, hydroxyapatite) were used instead of the powders obtained in Examples 1 and 2. The results are shown in Table 4.

TABLE 4

| | Residual Nucleic Acid | Nucleic Acid Removal |
|---|---|---|
| Sample | Concentration (mg/mL) | Efficiency (%) |
| CP1 | 0.007 | 83 |
| CP2 | 0.005 | 88 |
| Ca₃(PO₄)₂ | 0.015 | 62 |
| HA6 | 0.005 | 88 |
| Hydroxyapatite | 0.011 | 72 |

As shown in Table 4, the nucleic acid removal efficiencies in the cases of using the powders obtained in Example 1 (containing a polycation and a calcium salt) are superior to those in the cases of using calcium salts containing no polycation. That is, the dry powder obtained in Examples 1 and 2 were found to have high nucleic acid adsorbability.

### Example 5

### Polycation Elution Characteristic 1

The dry powders obtained in Examples 1 and 2 were suspended in buffer A or buffer B, followed by shaking at 37° C. for 2 hours. Buffers A and B have the following compositions. Those buffers are aqueous solutions each having a salt concentration corresponding to that of saliva. Buffer A: NaCl, 15 mmol; KCl, 20 mmol; NaHCO₃, 15 mmol; NaH₂PO₄, 13.2 mmol (pH 6.7); Buffer B: NaCl, 15 mmol; KCl, 20 mmol; NaHCO₃, 15 mmol (pH 8.4).

Thereafter, the suspensions were filtered with a filter having a pore size of 0.45 μm, and the concentrations of the polycations in the filtrate were determined by the ninhydrin method. The elution efficiencies were calculated from the following Formula 1 using measured values and the polycation contents (%) in the dry powder in Table 1. The results are shown in Table 5.
Elution efficiency (%)={(concentration of polycation in filtrate)×(volume of buffer)}÷{(polycation content in dry powder)×(weight of dry powder)}×100. Formula 1:

TABLE 5

| | | | | Filtrate | |
|---|---|---|---|---|---|
| | Sample | Buffer | | Polycation | Elution |
| | Weight | | Volume | Concentration | Efficiency |
| Sample | (g) | Kind | (mL) | (ppm) | % |
| CP1 | 2.5 | A | 50 | 6 | 2.40 |
| CP2 | 2.5 | A | 50 | 5 | 1.67 |
| HA1 | 5 | A | 60 | 0 | 0.00 |
| HA5 | 5 | A | 60 | 0 | 0.00 |
| HA5 | 5 | B | 60 | 0 | 0.00 |
| HA6 | 5 | A | 60 | 0 | 0.00 |
| HA6 | 5 | B | 60 | 0 | 0.00 |

As shown in Table 5, all the samples prepared in Examples 1 and 2 were found to have low elution efficiencies (0 to 2.4%). That is, in an aqueous solution having a salt concentration corresponding to that of saliva, each sample is hard to elute a polycation and can retain the polycation.

### Example 6

### Polycation Elution Characteristic 2

The dry powder obtained in Examples 1 and 2 (approximately 200 mg) were added to 10 mL of 1 mol/L aqueous NaCl solution, followed by shaking at room temperature for 2 hours. Thereafter, parts of the suspensions were filtered with a filter having a pore size of 0.45 μm, and the concentrations of polycations in the filtrate were determined by HPLC.

The determined concentrations of polycations in the filtrate (actual measurement values) and the concentrations in the case that all polycation in a sample (dry powder) is eluted (theoretical figure) were substituted for the following formula 2 to determine elution efficiencies.
Elution efficiency (%)={elution concentration (actual measurement value)/elution concentration (theoretical figure)}×100. Formula 2:

The concentrations of the polycations were determined by HPLC using a column of TSK gel ODS-120T, an eluent of an aqueous solution of 9.2 mmol/L dipotassium hydrogen phosphate, 9.2 mmol/L sodium sulfate, and 8.0 v/v % acetonitrile (pH 3.4) under conditions of a temperature of 40° C. and a flow rate of 0.5 mL/minute. A method of monitoring the absorption peak at 215 nm in the ultraviolet region was adopted.

A standard curve was created using ε-polylysine as a standard substance, and the concentrations of ε-polylysine in eluates were determined. The experimental results are collectively shown in Table 6.

TABLE 6

| | Sample | Sample | Elution | Elution | Elution |
|---|---|---|---|---|---|
| | Polycation | weight | Concentration | Concentration | Efficiency |
| Sample | Content (%) | (mg) | (ppm) (Theoretical) | (ppm) (Actual) | (%) |
| CP1 | 0.5 | 202 | 101 | 108 | 107 |
| CP2 | 0.6 | 201 | 121 | 141 | 117 |
| HA6 | 0.6 | 200 | 120 | 121 | 101 |

As shown in Table 6, approximately 100% of a polycation was eluted by exchanging a polycation with Na ion in the 1 mol/L aqueous NaCl solution having a salt concentration significantly higher than that of the buffer used in Example 5 (having a salt concentration approximately equal to that of saliva). That is, it was found that a polycation is not immobilized by a covalent bond in the product of the invention.

What is claimed is:

1. A product comprising a polycation and a calcium salt, wherein the product is obtained by the steps of removing a solvent from a mixture containing a polycation, a poorly water-soluble calcium salt, and the solvent.

2. The product of claim 1, wherein the mixture is obtained by mixing a polycation, a water-soluble calcium salt, a compound to convert the water-soluble calcium salt into the poorly water-soluble calcium salt, and the solvent.

3. The product of claim 1, wherein the polycation is a free polycation.

4. The product of claim 1, wherein the polycation is a polyamino acid.

5. The product of claim 1, wherein the polycation is polylysine.

6. The product of claim 1, wherein the polycation is ε-polylysine.

7. A composition for removing an oral endotoxin, comprising a product obtained by the steps of removing a solvent from a mixture containing a polycation, a poorly water-soluble calcium salt, and the solvent.

8. A composition for removing an oral endotoxin, comprising the product of claim 1.

9. A composition for removing an oral endotoxin, comprising the product of claim 2.

10. A composition for removing an oral endotoxin, comprising the product of claim 3.

11. A composition for removing an oral endotoxin, comprising the product of claim 4.

12. A composition for removing an oral endotoxin, comprising the product of claim 5.

13. A composition for removing an oral endotoxin, comprising the product of claim 6.

14. A composition for introducing an adsorbed nucleic acid into a cell comprising a product obtained by the steps of removing a solvent from a mixture containing a polycation, a poorly water-soluble calcium salt, the solvent and adsorbing a nucleic acid.

15. A method for producing a composition for removing an oral endotoxin, comprising a step of formulating the composition comprising the product obtained by a step of removing a solvent from a mixture containing a polycation, a poorly water-soluble calcium salt, and the solvent.

16. A method for producing a composition for introducing an adsorbed nucleic acid into a cell, comprising a step of formulating the composition comprising the product obtained by a step of removing a solvent from a mixture containing a polycation, a poorly water-soluble calcium salt, and the solvent.
